(19) United States
(12) Patent Application Publication
(10) Pub. No.: US 2011/0142942 A1
(43) Pub. Date: Jun. 16, 2011

(54) USE OF pH SENSITIVE COMPOUNDS IN TASTE MASKING OF DRUG SUBSTANCES WITHIN ORAL THIN FILM STRIPS

(75) Inventors: A. Mark Schobel, Whitehouse Station, NJ (US); Kevin Davidson, Valparaiso, IN (US); Laura Miloshoff, Schererville, IN (US); Pradeep Sanghvi, Schererville, IN (US); Madhu Hariharan, Munster, IN (US)

(73) Assignee: MONOSOL RX, LLC, Warren, NJ (US)

(21) Appl. No.: 12/965,196

(22) Filed: Dec. 10, 2010

Related U.S. Application Data
(60) Provisional application No. 61/285,301, filed on Dec. 10, 2009.

(51) Int. Cl. A61K 9/14; A61K 9/00

(52) U.S. Cl. 424/489; 424/400

(57) ABSTRACT

The present invention relates to an edible film dosage form that includes a film-forming polymer and a coated active composition capable of taste-masking an active contained therein. An edible film that includes an edible, water-soluble film forming polymer and an active with at least two coating layers is also disclosed.

### CROSS-REFERENCE TO RELATED APPLICATION

This application claims the benefit of U.S. Provisional Application No. 61/285,301, filed Dec. 10, 2009, the entire contents of which are incorporated herein by reference.

### FIELD OF THE INVENTION

The present invention relates to compositions relating to films containing active pharmaceutical agents. The invention more particularly relates to self-supporting dosage forms that include active components and pH sensitive components, which provide taste-masking for the active components. Some embodiments also include multiple coating layers of pH sensitive components.

### BACKGROUND OF THE RELATED TECHNOLOGY

While active agents such as pharmaceutical preparations may be included in a tablet or similar form to provide an accurate and consistent dose, such a form has several disadvantages in both the administration and preparation of the drug. Moreover, in such oral dosage forms, such as tablets or emulsions, pharmaceuticals have been coated to provide modified release. Particle sizes of particulate pharmaceuticals are not critical in such dosage forms and generally large particle sizes, i.e., greater than 200 microns have been used.

There have been several attempts to provide an alternate dosage form, such as a film that would include a pharmaceutical active. However, such attempts have not been successful in providing a film that incorporates a drug with sufficient uniformity to provide accurate dosing.

Moreover, due to the physical limitations on oral film dosages, e.g., relatively thin, small dosage units, the ability to deliver an active, such as a pharmaceutical, without the user experiencing the unpleasant taste of the active is extremely challenging. Such films typically dissolve in the mouth, leaving the active readily available for perception by the taste receptors.

Therefore, there is a need for therapeutic films, including orally ingestible films, which contain taste-masked active agents designed to overcome the problems associated with delivery of unpleasant tasting actives in film dosage forms.

### SUMMARY OF THE INVENTION

In one embodiment of the present invention, there is provided a film composition including: (i) a film-forming polymer; and (ii) an active composition including a granulated particle comprising at least one active selected from pharmaceutical agents, bioeffecting agents, bioactive agents, cosmeceuticals, nutraceuticals, vitamins, antigens, and such other actives, and combinations thereof, and a coating composition at least partially coating the active, said coating including a taste-masking effective amount of a reverse-enteric polymer composition and a water-insoluble polymeric composition, where the reverse-enteric polymer composition and the water-insoluble polymeric composition are present in an amount of about 9:1 to about 1:9 by weight of the coating composition and the at least partially coated active component is substantially water-insoluble at a neutral pH.

In another embodiment, there is provided an edible film for delivery of an active including, an edible film dosage form including: (a) an edible, water-soluble film forming polymer; and (b) an active composition including: (i) an active component selected from cosmetic agents, pharmaceutical agents, vitamins, antigens, bioactive agents, bioeffecting agents and combinations thereof; (ii) a first coating layer substantially surrounding the active component; and (iii) a second coating layer substantially surrounding the first coating layer; where the edible film dosage form is self-supporting.

### DETAILED DESCRIPTION OF THE PREFERRED EMBODIMENTS

The present invention provides a therapeutic film composition film for ingestion or topical administration, including a composition having a uniformly distributed combination of a polymer, a polar solvent, and a taste-masked active ingredient. The composition in its dried film form maintains the uniform distribution of components of which it was formed.

The therapeutic film dosage composition preferably includes a polymeric carrier matrix. Any desired polymeric carrier matrix may be used, provided that it is orally dissolvable and is suitable for use with humans, i.e., ingestion, implantation or topical use. The films are designed to dissolve when placed in contact with mucosal fluids, such as saliva, but the taste-masked active coating remains undissolved in order to protect the active and to prevent the user from detecting the taste of the active. Rapid release, controlled and sustained release compositions are among the various embodiments contemplated.

The film products of the invention may be produced by forming a matrix comprising at least one film-forming polymer and a polar solvent, optionally including other fillers known in the art. The active composition may be added during formation of the matrix, but is desirably added after the matrix is prepared to prevent the active from over exposure to the solvent. The solvent may be water, a polar organic solvent including, but not limited to, ethanol, isopropanol, acetone, methylene chloride, or any combination thereof. In some embodiments, the composition may employ little or no solvent, such as when hot melt extrusion processes are used. The film may be prepared by utilizing a casting or deposition methods and a controlled drying process or by various extrusion methods including hot melt extrusion. In the case of wet-coating, the film may be prepared through controlled drying processes, which include application of heat and/or radiation energy to the wet film matrix to form a visco-elastic structure, thereby controlling the uniformity of content of the film. Such processes are described in more detail in commonly assigned U.S. Pat. No. 7,425,292, the contents of which are incorporated herein by reference in their entirety. Alternatively, the films may be extruded as described in commonly assigned U.S. application Ser. No. 10/856,176, filed on May 28, 2004, and published as U.S. Patent Publication No. 2005/0037055 A1, the contents of which are incorporated herein by reference in their entirety. Desirably, the drying process locks-in the content uniformity of the film by forming a visco-elastic matrix with the first 4 to 10 minutes of drying.

The polymers that form the matrix of the film, i.e., the film-forming polymers, may be water-soluble, water-swellable, water-insoluble, or a combination of one or more either water-soluble, water-swellable or water-insoluble polymers. The polymer may include cellulose or a cellulose derivative. Specific examples of useful water-soluble polymers include, but are not limited to, polyethylene oxide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water-insoluble polymers include, but are not limited to, ethyl cellulose, hydroxypropyl ethyl cellulose, cellulose acetate phthalate, cellulose acetate, hydroxypropyl methyl cellulose phthalate and combinations thereof.

As used herein the phrase “water-soluble polymer” and variants thereof refer to a polymer that is at least partially soluble in water, and desirably fully or predominantly soluble in water, or absorbs water. Polymers that absorb water are often referred to as being water-swellable polymers. The materials useful with the present invention may be water-soluble or water-swellable at room temperature and other temperatures, such as temperatures exceeding room temperature. Moreover, the materials may be water-soluble or water-swellable at pressures less than atmospheric pressure. Desirably, the water-soluble polymers are water-soluble or water-swellable having at least 20 percent by weight water uptake. Water-swellable polymers having a 25 or greater percent by weight water uptake are also useful. In some embodiments, films formed from such water-soluble polymers may be sufficiently water-soluble to be dissolvable upon contact with bodily fluids.

Other film-forming polymers useful for incorporation into the films include biodegradable polymers, copolymers, block polymers and combinations thereof. Among the known useful polymers or polymer classes which meet the above criteria are: poly(glycolic acid) (PGA), poly(lactic acid) (PLA), polydioxanoes, polyoxalates, poly(α-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100 L, believed to be 100% lactide having a melting point within the range of 338°-347° F. (170°-175° C.); lactide/glycolide 100 L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.). The Biodel materials represent a family of various polyanhydrides which differ chemically.

Although a variety of different polymers may be used, it is desired to select polymers that provide mucoadhesive properties to the film, as well as a desired dissolution and/or disintegration rate. In particular, the time period for which it is desired to maintain the film in contact with the mucosal tissue depends on the type of active contained in the second delivery vehicle. Some actives may only require a few minutes for delivery through the mucosal tissue, whereas other actives may require up to several hours or even longer. Accordingly, in some embodiments, one or more water-soluble polymers, as described above, may be used to form the film. In other embodiments, however, it may be desirable to use combinations of water-soluble polymers and polymers that are water-swellable, water-insoluble and/or biodegradable, as provided above. The inclusion of one or more polymers that are water-swellable, water-insoluble and/or biodegradable may provide films with slower dissolution or disintegration rates than films formed from water-soluble polymers alone. As such, the film may adhere to the mucosal tissue for longer periods or time, such as up to several hours, which may be desirable for delivery of certain active components.

Oral dissolving films may be defined as falling into three main classes: fast dissolving, moderately slow dissolving and slow dissolving. Fast dissolving films generally dissolve in about 1 second to about 30 seconds. Moderately slow dissolving films generally dissolve in about 1 to about 30 minutes, and slow dissolving films generally dissolve in more than 30 minutes. Fast dissolving films may consist of low molecular weight hydrophilic polymers (i.e., polymers having a molecular weight between about 1,000 to 9,000). In contrast, slow dissolving films generally have high molecular weight polymers (i.e., having a molecular weight in the millions).

Moderately slow dissolving films tend to fall in between the fast and slow dissolving films. Moderate dissolving films dissolve rather quickly, but also have a good level of mucoadhesion. Moderate films are also flexible, quickly wettable, and are typically non-irritating to the user. For the instant invention, it is preferable to use films that fall between the categories of fast dissolving and moderate dissolving. Such films provide a quick enough dissolution rate (between about 1 minute and about 5 minutes), while providing an acceptable mucoadhesion level such that the film is not easily removable once it is placed in the oral cavity of the user.

Desirably, the individual film dosage has a small size, which is between about 0.5-1 inch by about 0.25-1.5 inch. Most preferably, the film dosage is about 0.75 inches by about 0.5 inches. The film dosage should have good adhesion when placed in the buccal cavity or in the sublingual region of the user. Further, the film dosage should disperse and dissolve at a moderate rate, that is, between about 1 minute to about 30 minutes, and most desirably between about 10 minutes and about 20 minutes. In some embodiments, however, it may be desired to allow the individual film dosage to dissolve slower, over a period of longer than about 30 minutes. In such slow dissolving embodiments, it is preferable that the film dosage has strong mucoadhesion properties. Sublingual and buccal films are contemplated and the size and thickness as well as the specific taste-masking composition and film matrix composition may be tailored to achieve the desired dissolution rate and time.

The polymer may be water soluble, water swellable, water insoluble or a combination of one or more either water soluble, water swellable or water insoluble polymers. The polymer may include cellulose or a cellulose derivative. Specific examples of useful water soluble polymers include, but are not limited to, polyethylene oxide (PEO), pullulan, hydroxypropylmethyl cellulose (HPMC), hydroxyethyl cellulose (HPC), hydroxypropyl cellulose, polydextrose, polyvinyl pyrrolidone, copolyvidone (vinylpyrrolidone/vinyl acetate copolymer), carboxymethyl cellulose, polyvinyl alcohol, sodium alginate, propylene glycol alginate, carrageenan, polyethylene glycol, xanthan gum, tragancanth gum, guar gum, acacia gum, arabic gum, polyacrylic acid, methylmethacrylate copolymer, poloxamer polymers, copolymers of acrylic acid and alkyl acrylate (available as Pemulen® polymers), carboxyvinyl copolymers, starch, gelatin, pectin, and combinations thereof.

As used herein the phrase “water soluble polymer” and variants thereof refer to a polymer that is at least partially soluble in water, and desirably fully or predominantly soluble in water, or absorbs water. Polymers that absorb water are often referred to as being water swellable polymers. The materials useful with the present invention may be water soluble or water swellable at room temperature and other temperatures, such as temperatures exceeding room temperature. Moreover, the materials may be water soluble or water swellable at pressures less than atmospheric pressure. Desirably, the water soluble polymers are water soluble or water swellable having at least 20 percent by weight water uptake. Water swellable polymers having a 25 or greater percent by weight water uptake are also useful. Films or dosage forms of the present invention formed from such water soluble polymers are desirably sufficiently water soluble to be dissolvable upon contact with bodily fluids.

Specific examples of useful water insoluble polymers include, but are not limited to, ethyl cellulose, hydroxypropyl ethyl cellulose, cellulose acetate phthalate, cellulose acetate, hydroxypropyl methyl cellulose phthalate, polyvinyl acetate phthalate, acrylic polymers, vinyl acetate, sodium sulphonated polyesters, carboxylated acrylics, trimethylpentanediol/adipic acid/glycerin cross polymer, polyglycerol-2-diisostearate/IPDI copolymer, carboxylated vinyl acetate copolymer, vinylpyrrolicone/vinyl acetate/alkylaminoacrylate terpolymers, and combinations thereof.

Other polymers useful for incorporation into the films of the present invention include biodegradable polymers, copolymers, block polymers and combinations thereof. Among the known useful polymers or polymer classes which meet the above criteria are: poly(glycolic acid) (PGA), poly(lactic acid) (PLA), polydioxanoes, polyoxalates, poly(α-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the DuPont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100 L, believed to be 100% lactide having a melting point within the range of 338°-347° F. (170°-175° C.); lactide/glycolide 100 L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.).

The Biodel materials represent a family of various polyanhydrides which differ chemically.

Although a variety of different polymers may be used, it is desired to select polymers to provide a desired viscosity of the mixture prior to drying. For example, if the active or other components are not soluble in the selected solvent, a polymer that will provide a greater viscosity is desired to assist in maintaining uniformity. On the other hand, if the components are soluble in the solvent, a polymer that provides a lower viscosity may be preferred.

The polymer plays an important role in affecting the viscosity of the film. Viscosity is one property of a liquid that controls the stability of the active in an emulsion, a colloid or a suspension. Generally the viscosity of the matrix will vary from about 400 cps to about 100,000 cps, preferably from about 800 cps to about 60,000 cps, and most preferably from about 1,000 cps to about 40,000 cps. Desirably, the viscosity of the film-forming matrix will rapidly increase upon initiation of the drying process.

The viscosity may be adjusted based on the selected active depending on the other components within the matrix. For example, if the component is not soluble within the selected solvent, a proper viscosity may be selected to prevent the component from settling which would adversely affect the uniformity of the resulting film. The viscosity may be adjusted in different ways. To increase viscosity of the film matrix, the polymer may be chosen of a higher molecular weight or crosslinkers may be added, such as salts of calcium, sodium and potassium. The viscosity may also be adjusted by adjusting the temperature or by adding a viscosity increasing component. Components that will increase the viscosity or stabilize the emulsion/suspension include higher molecular weight polymers and polysaccharides and gums, which include without limitation, alginate, carrageenan, hydroxypropyl methyl cellulose, locust bean gum, guar gum, xanthan gum, dextran, gum arabic, gellan gum and combinations thereof. Further adjustments may be made by changing the concentration of polymer used in the formulation or changing the total percentage of solids used in the formulation.

It has also been observed that certain polymers which when used alone would ordinarily require a plasticizer to achieve a flexible film, can be combined without a plasticizer and yet achieve flexible films. For example, HPMC or HPC when used in combination with PEO provide a flexible, strong film with the appropriate plasticity and elasticity for manufacturing and storage. No additional plasticizer or polyalcohol is needed for flexibility.

Additionally, polyethylene oxide (PEO), when used alone or in combination with at least one additional polymer, achieves flexible, strong films. Additional plasticizers or polyalcohols are not needed for flexibility. Non-limiting examples of suitable cellulosic polymers for combination with PEO include HPC and HPMC. PEO and HPC have essentially no gelation temperature, while HPMC has a gelation temperature of 58-64° C. (Methocel EF available from Dow Chemical Co.). Moreover, these films are sufficiently flexible even when substantially free of organic solvents, which may be removed without compromising film properties. As such, if there is no solvent present, then there is no plasticizer in the films. PEO based films also exhibit good resistance to tearing, little or no curling, and fast dissolution rates when the polymer component contains appropriate levels of PEO.

To achieve the desired film properties, the level and/or molecular weight of PEO in the polymer component may be varied. Modifying the PEO content affects properties such as tear resistance, dissolution rate, and adhesion tendencies. Thus, one method for controlling film properties is to modify the PEO content. For instance, in some embodiments rapid dissolving films are desirable. By modifying the content of the PEO polymer component, the desired dissolution characteristics can be achieved.

In accordance with the present invention, PEO desirably ranges from about 5% to about 100% by weight in the polymer component, more specifically in the amount of about 20% to about 100% by weight, even more specifically, in the amount of about 30% to about 70% by weight. In some embodiments, the PEO is present in the amount of about 40% to about 60% by weight of the polymer component. In some embodiments, the amount of PEO desirably ranges from about 1 mg to about 200 mg. The hydrophilic cellulosic polymer ranges from about 0% to about 80% by weight, more specifically, in the amount of about 30% to about 70% by weight, even more specifically, from about 40% to about 60% by weight of the polymer component, or in a ratio of up to about 4:1 with the PEO, and desirably in a ratio of about 1:1.

In some embodiments, it may be desirable to vary the PEO levels to promote certain film properties. To obtain films with high tear resistance and fast dissolution rates, levels of about 50% or greater of PEO in the polymer component are desirable. To achieve adhesion prevention, i.e., preventing the film from adhering to the roof of the mouth, PEO levels of about 20% to 75% are desirable. In some embodiments, however, adhesion to the roof of the mouth may be desired, such as for administration to animals or children. In such cases, higher levels of PEO may be employed. More specifically, structural integrity and dissolution of the film can be controlled such that the film can adhere to mucosa and be readily removed, or adhere more firmly and be difficult to remove, depending on the intended use.

The molecular weight of the PEO may also be varied. High molecular weight PEO, such as about 4 million, may be desired to increase mucoadhesiveness of the film. More desirably, the molecular weight may range from about 100,000 to 900,000, more desirably from about 100,000 to 600,000, and most desirably from about 100,000 to 300,000. In some embodiments, it may be desirable to combine high molecular weight (600,000 to 900,000) with low molecular weight (100,000 to 300,000) PEOs in the polymer component.

For instance, certain film properties, such as fast dissolution rates and high tear resistance, may be attained by combining small amounts of high molecular weight PEOs with larger amounts of lower molecular weight PEOs. Desirably, such compositions contain about 60% or greater levels of the lower molecular weight PEO in the PEO-blend polymer component.

To balance the properties of adhesion prevention, fast dissolution rate, and good tear resistance, desirable film compositions may include about 50% to 75% low molecular weight PEO, optionally combined with a small amount of a higher molecular weight PEO, with the remainder of the polymer component containing a hydrophilic cellulosic polymer (HPC or HPMC).

In some embodiments, the film may include polyvinyl alcohol (PVA), alone or in combination with at least one additional polymer. Examples of an additional polymer include a cellulosic polymer, starch, polyvinyl pyrrolidone (PVP), polyethylene oxide (PEO), an alginate, a pectin, or combinations thereof. PVA can be used in the films to improve film strength and/or to vary and slow dissolution times. The films are especially useful for the delivery of cosmetics, nutraceuticals and pharmaceuticals. In a preferred embodiment, the film includes PVA without any added platicizers. For example, the film can include both PVA, which provides strength to the film and PEO, which provides flexibility to the film and may obviate the need for a plasticizer.

PVA can be used in varying amounts depending upon the product application and characteristics desired. For example, in general, a larger amount of PVA will increase film strength and increase dissolution time. For films that require high active dosing, PVA can be used effectively at minimum amount of 0.5, preferably 1%, more preferably 5%, by weight of the film, to improve film strength. The PVA can be effectively used at a maximum amount of, for example, 80%, preferably 50%, more preferably 25% by weight of the film. For slowing dissolution time, PVA can be used at levels as high as 80%. A film containing an active can be coated on one or both surfaces with a PVA containing layer to modify the dissolution of the film and the release of an active from the film.

High loading of actives can decrease the strength and flexibility of the film. Including PVA in the film, either alone or in combination with at least one other polymer can increase the tensile strength of the film. Also, drug particles or taste-masked or coated or modified release drug particles may have a larger particle size, which can make loading of these particles into the film difficult. PVA can increase the viscosity of the film solution to allow improved drug loading.

The films may include a coated active composition to provide a taste masking of the active component. For example, the films may include ionic exchange resins, including but not limited to a water-insoluble organic or inorganic matrix material having covalently bound functional groups that are ionic or capable of being ionized under appropriate conditions. The organic matrix may be synthetic (e.g., polymers or copolymers or acrylic acid, methacrylic acid, sulfonated styrene or sulfonated divinylbenzene) or partially synthetic (e.g., modified cellulose or dextrans). The inorganic matrix may be, for example, silica gel modified by the addition of ionic groups. Most ion exchange resins are cross-linked by a crosslinking agent, such as divinylbenzene.

The coated active composition may include a taste-masking effective amount of a reverse-enteric polymer and a water-insoluble polymeric composition. Examples of reverse-enteric polymers may include copolymers of dimethyl aminoethyl methacrylate and neutral methacrylic acid esters, such as Eudragit® E-100 as sold by Evonik Industries and water-insoluble, pH independent base polymeric constituent, such as cellulose acetate or ethylcellulose, applied from an organic solution, such as acetone, on to the drug particles or granules in a fluid bed dryer using a top-spray, bottom-spray or Wurster column bottom spray configuration. Examples of water-insoluble polymers may include any as discussed above.

The relative portion of dimethyl aminoethyl methacrylate and neutral methacrylic acid esters to cellulose acetate may be in the range of about 9:1 to about 1:9. In some embodiments, the ratio may be in the range of about 4:1 to about 1:4. In other embodiments, the ratio may be the range of about 2:1 to about 1:2. In other embodiments, the ratio may be about 1:1. More typically, a range of 3:7 to about 7:3. In some embodiments, 100% E-100 can also be used. The actual ratio used depends on the degree of taste-masking required as well as the rate at which drug release is desired under acidic, neutral or basic conditions. In addition, the rate may vary depending on the solubility and other characteristics of the API being taste-masked.

The taste-masking coating may avoid or minimize the release of the drug from the particle in the manufacturing process of the oral thin film as well as within the neutral or near-neutral environment presented by the saliva of the oral cavity when the oral thin film containing the drug particle is administered to the consumer. The insoluble cellulose acetate along with the conditionally soluble reverse-enteric polymer in the coating serves as a barrier to water during manufacturing and as a barrier to saliva during consumption of the dosage form by the consumer. Upon contact with an acidic environment, the reverse enteric polymer dissolves to form pores in the overall coating structure thereby allowing the diffusion of drug out of the particle to be absorbed in the gastrointestinal tract.

In some embodiments, excipients may be added to the coating composition to further increase the rate of release of the drug from the film. Desirably, the taste-masking properties are still maintained after the addition of these excipients. One example of useful excipients for use in oral film dosages is an acid-reactive material, such as calcium carbonate or calcium phosphate. Alternatively, other acid-reactive materials, e.g., bases, may be employed to maintain the pH at levels which promote insolubility in the mouth, yet are readily soluble in acid pH once fully ingested.

Some of the useful taste-mask coating materials may swell in water regardless of the pH. For example, Eudragit® E-100, a polyacrylate polymer, may swell when placed in water regardless of the pH. Such water absorption and swelling of the polymer enhances the risk of the active to diffuse through the coating and which would defeat the taste-masking effect. One aspect of the invention includes a means to minimize diffusion of the drug through the coating, thereby preventing the perception of a bad-tasting active. This can be achieved by adjusting the ratio of the polymers in the coating. While adjusting the ratio of the polymers in the coating can be used to minimize the risk of active diffusing therethrough, the dissolution rate in the stomach may be lengthened as a result. This problem may be solved through the incorporation of water-insoluble, acid-reactive materials such as calcium carbonate, incorporated into the taste-masking composition. When the pH of the microenvironment in the polymer layer is neutral, e.g., in the mouth, the acid-reactive material is unreactive and insoluble. Once the taste-masking particles come into contact with gastric acid, the acid-reactive material, e.g., calcium carbonate would react with gastric acid to liberate carbon dioxide. In this way, the effervescing action of the carbon dioxide selectively disrupts the coating layer in the presence of stomach acid and facilitates dissolution, release and absorption of the active.

Particle size of the acid-reactive material also plays a role in facilitating coating disruption. In some embodiments, for example, calcium carbonate may have a larger reactive surface area and produce higher amounts of carbon dioxide, thus enhancing disruption of the coating. In some embodiments, the particle size of the acid-reactive material, e.g., calcium carbonate, may be from about 0.5 um to about 25 um. In some embodiments, in some embodiments, the particle size may be from about 1.0 um to about 15 um. In some embodiments, particles may range from 1 um to 10 um. In some embodiments, the particle size may be from about 5.0 um to about 10 um. Acid-reactive materials may be used in the granulation process, taste-masking process or both.

In some embodiments, the oral thin film may include non-pH dependent materials such as sucrose, natural sweeteners or artificial sweeteners, surfactants, fillers, coloring agents, flavors, disintegrating agents, salts and other non-pH dependent materials, disintegration enhancers and combinations thereof. In these embodiments, the non-pH dependent materials may be released as the polymer layer is increasingly hydrated and swells. This further increases the permeability of the coating by forming contiguous channels within the coating through which the drug can diffuse out of the particulate core.

In other embodiments of the invention, insoluble and hydrophobic materials may be added to the polymer composition in the taste-masking layer to help in making the particle hydrophobic and resist penetration by water during manufacturing. Such components may have the added benefit of improving processiblity during fluid bed coating by alleviating the build-up of electrostatic charges which may cause improper application of the coating on the particles. Examples of compounds that may be used in this capacity may include magnesium stearate, stearic acid, sodium stearyl fumarate or talc, silicon dioxide and combinations thereof.

Plasticizers such as triacetin, dibutyl sebacate and triethyl citrate and diethyl phthalate may also be added to improve the properties of the taste masking coating.

In some embodiments, a two-layered film approach is provided to further improve the functionality of the reverse-enteric polymer coating system for taste-masking. The finished coated particle may be dispersed in an aqueous polymer solution to be cast into oral thin films. The coated particle may then be exposed to water in an aqueous polymer solution during the manufacturing process for up to an hour, or more.

During this process, the reverse-enteric polymer may retain its integrity but will absorb water and swell. Certain therapeutic actives, such as pharmaceutical agents with high diffusion rates, may diffuse to a significant extent through the swollen polymer layer, causing premature release of the active into the oral thin film, and resulting in a bitter or otherwise unpleasant sensation during oral consumption.

To minimize the amount of possible diffusion, a dual-coat method is used in which an ethylcelluose based coating solution (“under-coat”) is sprayed onto the core particle/granule containing the active. A “top-coat” is applied over the under-coat, the top coating being made from reverse-enteric polymer composition. The ethylcellulose layer is relatively insoluble in water, does not swell appreciably, and creates a temporary moisture barrier against premature saliva penetration through the top coat. When the particle is exposed to neutral pH, the outer top-coat may swell as it absorbs water, but the water will be prevented from reaching the active due to the under-coat.

Once swallowed and exposed to acidic medium, the top-coat dissolves and the ethylcellulose under-coat is now completely exposed to water, which in turn causes release of the drug from the core. The top-coat in this case may include similar additional non-pH dependent additives as described above.

The under-coat thus acts as a temporary moisture barrier during the time the coated particles are sitting in the aqueous neutral environment, e.g., during manufacturing or while residing in the mouth, to prevent leaching of the drug and concomitant unpleasant taste.

In some embodiments a scavenger or other complexing agent, for the active is incorporated into the top coat, under coat or granulated with the active. Scavengers can attract actives, such as drug molecules and other charged species, and sequester them. For example, charged drug molecules may be attracted to the interstitial spaces of finely divided magnesium trisilicate. In this manner, the drug is removed or scavenged from the aqueous environment, thereby reducing or eliminating the possibility of the user tasting the drug. Other absorbate materials may also be used. Additionally, materials such as cyclodextrin, which can form inclusion complexes with drug members and other actives, may also be employed.

Such scavengers, absorbates and other similar agents may be employed in taste-masking effecting amounts. The amount of scavenger, absorbates or other similar agents may be dependent to the amount of free drug expected in the resulting film. For example, in some embodiments, they may be present in the range of about 1:10 to about 10:1 by weight of the free drug present in the film. In other embodiments, they may be present in the range of about 1:5 to about 5:1 by weight of the free drug present in the film. In other embodiments, they may be present in the range of about 1:3 to about 3:1 by weight of the free drug present in the film. In some embodiments, they may be present in a 1:1 ratio with the free drug present in the film.

Anti-foaming and/or de-foaming components may also be used with the films. These components aid in the removal of air, such as entrapped air, from the film-forming compositions. Such entrapped air may lead to non-uniform films. Simethicone is one particularly useful anti-foaming and/or de-foaming agent. The present invention, however, is not so limited and other suitable anti-foam and/or de-foaming agents may be used.

As a related matter, simethicone and related agents may be employed for densification purposes. More specifically, such agents may facilitate the removal of voids, air, moisture, and similar undesired components, thereby providing denser, and thus more uniform films. Agents or components which perform this function can be referred to as densification or densifying agents. As described above, entrapped air or undesired components may lead to non-uniform films.

Simethicone is generally used in the medical field as a treatment for gas or colic in babies. Simethicone is a mixture of fully methylated linear siloxane polymers containing repeating units of polydimethylsiloxane which is stabilized with trimethylsiloxy end-blocking unites, and silicon dioxide. It usually contains 90.5-99% polymethylsiloxane and 4-7% silicon dioxide. The mixture is a gray, translucent, viscous fluid which is insoluble in water.

In order to prevent the formation of air bubbles in the films, the mixing step may be performed under vacuum. However, as soon as the mixing step is completed, and the film solution is returned to the normal atmosphere condition, air will be re-introduced into or contacted with the mixture. In many cases, tiny air bubbles will be again trapped inside this polymeric viscous solution. The incorporation of simethicone into the film-forming composition either substantially reduces or eliminates the formation of air bubbles during and after mixing.

Any other optional components described in commonly assigned U.S. Pat. No. 7,425,292 and U.S. application Ser. No. 10/856,176, referred to above, also may be included in the films described herein.

The wet casting manufacturing process of oral thin films requires that the actives are suspended in an aqueous solution or solvent for at least a minute. In some instances, the actives are suspended in the aqueous solution or solvent for at least two hours. Inclusion of bitter or bad-tasting active pharmaceutical agents in oral thin film requires that a coating be applied to the active to prevent the consumer from experiencing bad taste. This coating acts as a barrier that prevents access of a solvent, such as water, to the active. In addition, the barrier must be practically water-insoluble at neutral pH conditions.

Aqueous polymer solutions employed in the invention may be formulated to have a pH greater than 6. In some instances, the aqueous polymer solution may be formulated to have pH of between about 5 and about 9.

A variety of optional components and fillers also may be added to the films. These may include, without limitation: surfactants; plasticizers; polyalcohols; anti-foaming agents, such as silicone-containing compounds, which promote a smoother film surface by releasing oxygen from the film; thermo-setting gels such as pectin, carageenan, and gelatin, which help in maintaining the dispersion of components; inclusion compounds, such as cyclodextrins and caged molecules; coloring agents; and flavors. In some embodiments, more than one active component may be included in the film.

Additives may be included in the films. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active agent(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; and other similar polymers.

Such extenders may optionally be added in any desired amount desirably within the range of up to about 80%, desirably about 3% to 50% and more desirably within the range of 3% to 20% based on the weight of all film components.

Further additives may be inorganic fillers, such as the oxides of magnesium aluminum, silicon, titanium, etc. desirably in a concentration range of about 0.02% to about 3% by weight and desirably about 0.02% to about 1% based on the weight of all film components.

Further examples of additives are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like, added in concentrations ranging from about 0.5% to about 30%, and desirably ranging from about 0.5% to about 20% based on the weight of the polymer.

There may further be added compounds to improve the flow properties of the starch material such as animal or vegetable fats, desirably in their hydrogenated form, especially those which are solid at room temperature. These fats desirably have a melting point of 50° C. or higher. Preferred are tri-glycerides with C₁₂-, C₁₄-, C₁₆-, C₁₈-, C₂₀- and C₂₂-fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e. with C₁₂-, C₁₄-, C₁₆-, C₁₈-, C₂₀- and C₂₂-fatty acids. The total amounts used of the fats, mono-, di-glycerides and/or lecithins may be up to about 5% and preferably within the range of about 0.5% to about 2% by weight of the total film composition.

It further may be useful to add silicon dioxide, calcium silicate, or titanium dioxide in a concentration of about 0.02% to about 1% by weight of the total composition. These compounds act as texturizing agents.

Lecithin is one surface active agent for use in the films described herein. Lecithin may be included in the feedstock in an amount of from about 0.25% to about 2.00% by weight. Other surface active agents, i.e. surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL which is commercially available from BASF, are also useful. Carbowax™ is yet another modifier which is very useful in the present invention. Tweens™ or combinations of surface active agents may be used to achieve the desired hydrophilic-lipophilic balance (“HLB”).

Other ingredients include binders which contribute to the ease of formation and general quality of the films. Non-limiting examples of binders include starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols. If desired, the film may include other additives, such as keratin, or proteins, including proteins that are useful in forming a gel, such as gelatine.

Further potential additives include solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable actives may fit within the hydrophobic cavity, thereby producing an inclusion complex, which is soluble in water. Accordingly, the formation of the inclusion complex permits very insoluble and/or instable actives to be dissolved in water. A particularly desirable example of such agents are cyclodextrins, which are cyclic carbohydrates derived from starch. Other similar substances, however, are considered well within the scope of the present invention.

Suitable coloring agents include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide.

Other examples of coloring agents include known azo dyes, organic or inorganic pigments, or coloring agents of natural origin. Inorganic pigments are preferred, such as the oxides of iron or titanium, these oxides, being added in concentrations ranging from about 0.001 to about 10%, and preferably about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors.

Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), d-Limonene (citrus), Dimethyl anthranilate (grape) combinations thereof and the like.

The sweeteners may be chosen from the following non-limiting list: glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

### EXAMPLES

### Example 1

### Composition of a Dual Coat Particle

The components of the dual coat film are listed in Table 1 along with the percentages of each component by weight of the coated active particle.

TABLE 1

| Composition of Dual Coated | |
|---|---|
| Active Particle | Weight % |
| Active ingredient | 46.44 |
| Granulation ingredients | 4.85 |
| Under Coat (moisture barrier) | 15.38 |
| ingredients | |
| Top Coat (taste mask) ingredients | 33.33 |
| TOTAL | 100 |

The components of the film dosage unit are listed in Table 2 along with the percentages of each component by weight of the coated active particle.

TABLE 2

| Composition of Film Dosage Unit | Weight % |
|---|---|
| Dual Coated Active Particle | 33.65 |
| Water Soluble Polymers | 33.26 |
| Fillers | 9.45 |
| Colorants | 0.54 |
| Sweeteners | 6.50 |
| Flavors | 10.60 |
| Sequestering Agents | 6.00 |
| TOTAL | 100 |

As a result of the example set forth above a coherent, accurate coated particle is produced which is placed into a polymer matrix to form individual film dosage units which dissolves substantially instantaneously in the oral cavity, releasing all components substantially simultaneously. The coated active composition will remain coated until it reaches pH level of approximately 4 and thus is substantially undetectable in the oral cavity.

Thus, while there have been described what are presently believed to be the preferred embodiments of the present invention, those skilled in the art will realize that other and further embodiments may result using the present invention, and it is intended to include all such further embodiments as come within the true scope of the invention as outlined in the appended claims.

What is claimed is:

1. A therapeutic film dosage comprising:
a film composition comprising:
(i) a film-forming polymer; and
(ii) a coated active composition comprising at least one particulate active and a coating composition comprising a taste-masking effective amount of a reverse-enteric polymer composition and a water-insoluble polymeric composition, wherein said reverse-enteric polymer composition and said water-insoluble polymeric composition are present in an amount of about 9:1 to about 1:9 by weight of the coating composition,
wherein said coating composition at least partially surrounds said active component and the at least partially coated active component is water-insoluble at a neutral pH.

2. The edible film dosage form of claim 1, wherein said coating composition prevents water from contacting said active component.

3. The edible film dosage form of claim 1, wherein said coating composition substantially prevents organoleptic detection of the active in the mouth.

4. The edible film dosage form of claim 1, wherein said coating composition is water-insoluble in the pH range of about 5 to about 9.

5. The edible film dosage form of claim 1, wherein said coating composition is water-soluble in the pH range of about 1 to about 4.5.

6. The edible film dosage form of claim 1, wherein said coating composition is substantially water-insoluble for about one minute to about two hours.

7. The edible film dosage form of claim 1, wherein said reverse-enteric polymer composition is selected from the group consisting of dimethylaminoethyl methacrylate, neutral methacrylic acid esters and combinations thereof.

8. The edible film dosage form of claim 7, wherein said water-insoluble polymeric composition is selected from the group consisting of cellulose acetate, ethylcellulose, hydroxypropyl ethyl cellulose, cellulose acetate phthalate, hydroxypropyl methyl cellulose phthalate, polyvinyl acetate phthalate and combinations thereof.

9. The edible film dosage form of claim 1, wherein said coating composition further comprises an acid-reactive component.

10. The edible film dosage form of claim 9, wherein said acid-reactive material is selected from the group consisting of calcium carbonate, calcium phosphate and combinations thereof.

11. The edible film dosage form of claim 10, wherein said calcium carbonate has a particle size range of from about 0.5 μm to about 25 μm.

12. The edible film dosage form of claim 1, further comprising non-pH dependent materials selected from the group consisting of sucrose, natural sweeteners, artificial sweeteners, and combinations thereof.

13. The dosage form of claim 1, further comprising insoluble, hydrophobic materials selected from the group consisting of magnesium stearate, stearic acid, sodium stearyl fumarate, and combinations thereof.

14. The therapeutic dosage form of claim 1, wherein said particle is granulated.

15. The therapeutic dosage form of claim 14, wherein said granulated particle further comprises an active adsorbate comprising magnesium trisilicate.

16. The edible film dosage form of claim 1, wherein said coating composition comprises an active adsorbate comprising magnesium trisilicate.

17. A therapeutic film for delivery of an active comprising:
an edible film dosage comprising:
(a) an edible, water-soluble film forming polymer; and
(b) an active composition comprising:
(i) an active component selected from the group consisting of cosmetic agents, pharmaceutical agents, vitamins, bioactive agents and combinations thereof;
(ii) a first coating layer substantially surrounding said active component; and
(iii) a second coating layer substantially surrounding said first coating layer;
wherein said edible film dosage form is self-supporting.

18. The edible film of claim 17, wherein said first coating layer comprises ethylcellulose.

19. The edible film of claim 17, wherein said first coating layer is water-insoluble.

20. The edible film of claim 17, wherein said second coating layer is water-insoluble in the pH range of about 5 to about 9.

21. The edible film of claim 17, wherein said second coating layer comprises a compound selected from the group consisting of dimethyl aminoethyl methacrylate, neutral methacrylic acid esters, or combinations thereof and a water-insoluble, pH-independent base polymeric constituent.

22. The edible film dosage form of claim 17, wherein said first coating composition comprises an active adsorbate comprising magnesium trisilicate.

23. The edible film dosage form of claim 17, wherein said second coating composition comprises an active adsorbate comprising magnesium trisilicate.
